(19) United States
(12) Patent Application Publication
(10) Pub. No.: US 2025/0282726 A1
(43) Pub. Date: Sep. 11, 2025

(54) 5-BROMO-2-HYDROXY-N'-[(1E)-1-(PYRIDIN-2-YL) ETHYLIDENE]BENZOHYDRAZIDE AS AN ANTI-CANCER AND ANTIMICROBIAL AGENT

(71) Applicant: KING SAUD UNIVERSITY, Riyadh (SA)

(72) Inventors: MOHSIN KAZI, Riyadh (SA); MD ABDUL MAJED PATWARY, Riyadh (SA); SHARMIN AKTHER RUPA, Riyadh (SA); ASHOK KUMAR, Riyadh (SA); KHALID M. ALGHAMDI, Riyadh (SA)

(21) Appl. No.: 19/010,916

(22) Filed: Jan. 6, 2025

Related U.S. Application Data
(62) Division of application No. 18/601,445, filed on Mar. 11, 2024, now abandoned.

(51) Int. Cl. C07D 213/53; A61K 31/4402; A61P 31/04; A61P 31/10; A61P 35/00

(52) U.S. Cl. CPC C07D 213/53; A61K 31/4402; A61P 31/04; A61P 31/10; A61P 35/00

(57) ABSTRACT

A 5-bromo-2-hydroxy-N′-[(1E)-1-(pyridin-2-yl)ethylidene]benzohydrazide compound, its synthesis, and its use as an anticancer and an antimicrobial agent.

### CROSS-REFERENCE TO RELATED APPLICATION

This application is a divisional of U.S. patent application Ser. No. 18/601,445, filed on Mar. 11, 2024, the entire contents of which are incorporated herein by reference.

### BACKGROUND

### 1. Field

The present disclosure relates to the compound 5-bromo-2-hydroxy-N′-[(1E)-1-(pyridin-2-yl) ethylidene]benzohydrazide, its synthesis, and its use as an anti-cancer and antimicrobial agent.

### 2. Description of the Related Art

There remains an ongoing need for new therapeutically active agents for treating a variety of diseases, disorders, and conditions including, but not limited to, various forms of cancer, and the like.

Further, once such new therapeutically active agents are designed and/or discovered, they are often difficult to synthesize and/or prepare. It remains difficult to prepare such compounds without using solvents, to obtain a high yield, based on efficient reaction times and which are easy to use.

Thus, new molecules having desired therapeutic activities and solving the aforementioned problems are desired.

### SUMMARY

The present subject matter relates to a 5-bromo-2-hydroxy-N′-[(1E)-1-(pyridin-2-yl) ethylidene]benzohydrazide. The product can be acquired in exceptional yields (average about 82%) using a two-component reaction between methyl-5-bromosalicylic hydrazide in methanol and 2-acetylpyridine in a mixture of acetonitrile and methanol. The product can be analyzed using spectral data; IR, NMR & elemental analysis. The prepared compound can be used for its anticancer activity, which gave high efficiency.

In an embodiment, the present subject matter relates to a 5-bromo-2-hydroxy-N′-[(1E)-1-(pyridin-2-yl)ethylidene]benzohydrazide compound having the formula I:

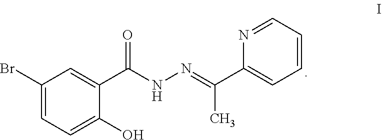 [SMILES: C/C(=N\NC(=O)c1cc(Br)ccc1O)c1ccccn1]

In another embodiment, the present subject matter relates to a pharmaceutically acceptable composition comprising a therapeutically effective amount of the 5-bromo-2-hydroxy-N′-[(1E)-1-(pyridin-2-yl)ethylidene]benzohydrazide compound and a pharmaceutically acceptable carrier.

In a further embodiment, the present subject matter relates to a method of treating cancer in a patient comprising administering to a patient in need thereof a therapeutically effective amount of the 5-bromo-2-hydroxy-N′-[(1E)-1-(pyridin-2-yl)ethylidene]benzohydrazide compound.

In an additional embodiment, the present subject matter relates to a method of treating a microbial infection in a patient comprising administering to a patient in need thereof a therapeutically effective amount of the 5-bromo-2-hydroxy-N′-[(1E)-1-(pyridin-2-yl)ethylidene]benzohydrazide compound.

In one more embodiment, the present subject matter relates to a method of making the 5-bromo-2-hydroxy-N′-[(1E)-1-(pyridin-2-yl)ethylidene]benzohydrazide compound, the method comprising: adding a solution of methyl-5-bromosalicylic hydrazide in methanol to a solution of 2-acetylpyridine in a mixture of acetonitrile and methanol to obtain a reaction mixture; stirring the reaction mixture; collecting a precipitate by filtration and drying under a vacuum; and obtaining the 5-bromo-2-hydroxy-N′-[(1E)-1-(pyridin-2-yl)ethylidene]benzohydrazide compound.

These and other features of the present subject matter will become readily apparent upon further review of the following specification.

### BRIEF DESCRIPTION OF THE DRAWINGS

FIG. 1 shows Diverse interaction of 5-bromo-2-hydroxy-N′-[(1E)-1-(pyridin-2-yl)ethylidene]benzohydrazide inside the human PDB ID: 4FM9 and 4YBJ active sites.

FIG. 2 shows the effects of different concentrations of 5-bromo-2-hydroxy-N′-[(1E)-1-(pyridin-2-yl)ethylidene]benzohydrazide compound as % cell viability on a liver cancer cell line as measured by MTT 72 hour following exposure.

FIG. 3 shows the effects of different concentrations of 5-bromo-2-hydroxy-N′-[(1E)-1-(pyridin-2-yl)ethylidene]benzohydrazide compound as % cell viability on a liver cancer cell line as measured by MTT 72 hour following exposure.

FIG. 4 shows the effects of different concentrations of 5-bromo-2-hydroxy-N′-[(1E)-1-(pyridin-2-yl)ethylidene]benzohydrazide compound as % cell viability on a liver cancer cell line as measured by MTT 72 hour following exposure.

FIG. 5 shows the effects of different concentrations of 5-bromo-2-hydroxy-N′-[(1E)-1-(pyridin-2-yl)ethylidene]benzohydrazide compound as % cell viability on a liver cancer cell line as measured by MTT 72 hour following exposure.

### DETAILED DESCRIPTION OF THE PREFERRED EMBODIMENTS

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

It will be understood by those skilled in the art with respect to any chemical group containing one or more substituents that such groups are not intended to introduce any substitution or substitution patterns that are sterically impractical and/or physically non-feasible.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

“Subject” as used herein refers to any animal classified as a mammal, including humans, domestic and farm animals, and zoo, sports, and pet companion animals such as household pets and other domesticated animals such as, but not limited to, cattle, sheep, ferrets, swine, horses, poultry, rabbits, goats, dogs, cats and the like.

“Patient” as used herein refers to a subject in need of treatment of a condition, disorder, or disease, such as cancer and microbial infections.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The present subject matter relates to a 5-bromo-2-hydroxy-N′-[(1E)-1-(pyridin-2yl)ethylidene]benzohydrazide. The product can be acquired in exceptional yields (average about 82%) using a two-component reaction between methyl-5-bromosalicylic hydrazide in methanol and 2-acetylpyridine in a mixture of acetonitrile and methanol. The product can be analyzed using spectral data; IR, NMR & elemental analysis. The prepared compound can be used for its anticancer activity and/or antimicrobial activity, which gave high efficiency.

In an embodiment, the present subject matter relates to a 5-bromo-2-hydroxy-N′-[(1E)-1-(pyridin-2-yl)ethylidene]benzohydrazide compound having the formula I:

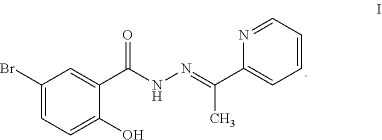 [SMILES: C/C(=N\NC(=O)c1cc(Br)ccc1O)c1ccccn1]

In various embodiments, the above compound may be defined or described as “compound L”.

In certain embodiments, the 5-bromo-2-hydroxy-N′-[(1E)-1-(pyridin-2-yl)ethylidene]benzohydrazide compound can be obtained as a white solid.

In another embodiment, the present subject matter relates to a pharmaceutically acceptable composition comprising a therapeutically effective amount of the 5-bromo-2-hydroxy-N′-[(1E)-1-(pyridin-2-yl)ethylidene]benzohydrazide compound and a pharmaceutically acceptable carrier.

In this regard, the present subject matter is further directed to pharmaceutical compositions comprising a therapeutically effective amount of the compound as described herein together with one or more pharmaceutically acceptable carriers, excipients, or vehicles. In some embodiments, the present compositions can be used for combination therapy, where other therapeutic and/or prophylactic ingredients can be included therein.

The present subject matter further relates to a pharmaceutical composition, which comprises a present compound together with at least one pharmaceutically acceptable auxiliary.

Non-limiting examples of suitable excipients, carriers, or vehicles useful herein include liquids such as water, saline, glycerol, polyethylene glycol, hyaluronic acid, ethanol, and the like. Suitable excipients for nonliquid formulations are also known to those of skill in the art. A thorough discussion of pharmaceutically acceptable excipients and salts useful herein is available in Remington's Pharmaceutical Sciences, 18th Edition. Easton, Pa., Mack Publishing Company, 1990, the entire contents of which are incorporated by reference herein.

The present compound is typically administered at a therapeutically or pharmaceutically effective dosage, e.g., a dosage sufficient to provide treatment for cancer or a microbial infection. Administration of the compound or pharmaceutical compositions thereof can be by any method that delivers the compound systemically and/or locally. These methods include oral routes, parenteral routes, intraduodenal routes, and the like.

While human dosage levels have yet to be optimized for the present compound, generally, a daily dose is from about 0.01 to 10.0 mg/kg of body weight, for example about 0.1 to 5.0 mg/kg of body weight. The precise effective amount will vary from subject to subject and will depend upon the species, age, the subject's size and health, the nature and extent of the condition being treated, recommendations of the treating physician, and the therapeutics or combination of therapeutics selected for administration. The subject may be administered as many doses as is required to reduce and/or alleviate the signs, symptoms, or causes of the disease or disorder in question, or bring about any other desired alteration of a biological system.

In employing the present compound for treatment of cancer, any pharmaceutically acceptable mode of administration can be used with other pharmaceutically acceptable excipients, including solid, semi-solid, liquid or aerosol dosage forms, such as, for example, tablets, capsules, powders, liquids, suspensions, suppositories, aerosols or the like. The present compounds can also be administered in sustained or controlled release dosage forms, including depot injections, osmotic pumps, pills, transdermal (including electrotransport) patches, and the like, for the prolonged administration of the compound at a predetermined rate, preferably in unit dosage forms suitable for single administration of precise dosages.

The present compounds may also be administered as compositions prepared as foods for humans or animals, including medical foods, functional food, special nutrition foods and dietary supplements. A “medical food” is a product prescribed by a physician that is intended for the specific dietary management of a disorder or health condition for which distinctive nutritional requirements exist and may include formulations fed through a feeding tube (referred to as enteral administration or gavage administration).

A “dietary supplement” shall mean a product that is intended to supplement the human diet and may be provided in the form of a pill, capsule, tablet, or like formulation. By way of non-limiting example, a dietary supplement may include one or more of the following dietary ingredients: vitamins, minerals, herbs, botanicals, amino acids, and dietary substances intended to supplement the diet by increasing total dietary intake, or a concentrate, metabolite, constituent, extract, or combinations of these ingredients, not intended as a conventional food or as the sole item of a meal or diet. Dietary supplements may also be incorporated into foodstuffs, such as functional foods designed to promote control of glucose levels. A “functional food” is an ordinary food that has one or more components or ingredients incorporated into it to give a specific medical or physiological benefit, other than a purely nutritional effect. “Special nutrition food” means ingredients designed for a particular diet related to conditions or to support treatment of nutritional deficiencies.

Generally, depending on the intended mode of administration, the pharmaceutically acceptable composition will contain about 0.1% to 90%, for example about 0.5% to 50%, by weight of the present compound, the remainder being suitable pharmaceutical excipients, carriers, etc.

One manner of administration for the conditions detailed above is oral, using a convenient daily dosage regimen which can be adjusted according to the degree of affliction. For such oral administration, a pharmaceutically acceptable, non-toxic composition is formed by the incorporation of any of the normally employed excipients, such as, for example, mannitol, lactose, starch, magnesium stearate, sodium saccharine, talcum, cellulose, sodium croscarmellose, glucose, gelatin, sucrose, magnesium carbonate, and the like. Such compositions take the form of solutions, suspensions, tablets, dispersible tablets, pills, capsules, powders, sustained release formulations and the like.

The present compositions may take the form of a pill or tablet and thus the composition may contain, along with the active ingredient, a diluent such as lactose, sucrose, dicalcium phosphate, or the like; a lubricant such as magnesium stearate or the like; and a binder such as starch, gum acacia, polyvinyl pyrrolidine, gelatin, cellulose and derivatives thereof, and the like.

Liquid pharmaceutically administrable compositions can, for example, be prepared by dissolving, dispersing, etc. an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, sodium acetate, sodium citrate, cyclodextrin derivatives, sorbitan monolaurate, triethanolamine acetate, triethanolamine oleate, etc.

For oral administration, a pharmaceutically acceptable non-toxic composition may be formed by the incorporation of any normally employed excipients, such as, for example, pharmaceutical grades of mannitol, lactose, starch, magnesium stearate, talcum, cellulose derivatives, sodium croscarmellose, glucose, sucrose, magnesium carbonate, sodium saccharin, talcum and the like. Such compositions take the form of solutions, suspensions, tablets, capsules, powders, sustained release formulations and the like.

For a solid dosage form, a solution or suspension in, for example, propylene carbonate, vegetable oils or triglycerides, may be encapsulated in a gelatin capsule. Such diester solutions, and the preparation and encapsulation thereof, are disclosed in U.S. Pat. Nos. 4,328,245; 4,409,239; and 4,410,545, the contents of each of which are incorporated herein by reference. For a liquid dosage form, the solution, e.g., in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and the like, and encapsulating these solutions or suspensions in hard or soft gelatin capsule shells.

Other useful formulations include those set forth in U.S. Pat. Nos. Re. 28,819 and 4,358,603, the contents of each of which are hereby incorporated by reference.

Another manner of administration is parenteral administration, generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents, solubility enhancers, and the like, such as for example, sodium acetate, sorbitan monolaurate, triethanolamine oleate, cyclodextrins, etc.

Another approach for parenteral administration employs the implantation of a slow-release or sustained-release system, such that a constant level of dosage is maintained. The percentage of active compound contained in such parenteral compositions is highly dependent on the specific nature thereof, as well as the activity of the compound and the needs of the subject. However, percentages of active ingredient of 0.01% to 10% in solution are employable and will be higher if the composition is a solid which will be subsequently diluted to the above percentages. The composition may comprise 0.2% to 2% of the active agent in solution.

Nasal solutions of the active compound alone or in combination with other pharmaceutically acceptable excipients can also be administered.

Formulations of the active compound or a salt may also be administered to the respiratory tract as an aerosol or solution for a nebulizer, or as a microfine powder for insufflation, alone or in combination with an inert carrier such as lactose. In such a case, the particles of the formulation have diameters of less than 50 microns, for example less than 10 microns.

In a further embodiment, the present subject matter relates to a method of treating cancer in a patient comprising administering to a patient in need thereof a therapeutically effective amount of the 5-bromo-2-hydroxy-N′-[(1E)-1-(pyridin-2-yl)ethylidene]benzohydrazide compound.

In certain embodiments in this regard, the cancer can be one or more selected from the group consisting of lung cancer and liver cancer.

In an additional embodiment, the present subject matter relates to a method of treating a microbial infection in a patient comprising administering to a patient in need thereof a therapeutically effective amount of the 5-bromo-2-hydroxy-N′-[(1E)-1-(pyridin-2-yl)ethylidene]benzohydrazide compound.

In certain embodiments in this regard, the microbial infection can be caused by one or more bacteria or fungi.

In an embodiment, the microbial infection can be caused by a gram positive bacteria. In this regard, non-limiting examples of the gram positive bacterial strains causing the microbial infection may include Staphylococcus aureus and Bacillus megaterium. In another embodiment, the microbial infection can be caused by one or more gram negative bacteria. In this regard, non-limiting examples of the one or more gram-negative bacterial strains causing the microbial infection include Salmonella typhi and Escherichia coli. In a further embodiment, the microbial infection can be caused by a fungus. In this regard, non-limiting examples of the fungus causing the microbial infection include Trichoderma harzianum and Aspergillus niger. Any combination of any of the foregoing are further contemplated herein.

In one more embodiment, the present subject matter relates to a method of making the 5-bromo-2-hydroxy-N′-[(1E)-1-(pyridin-2-yl)ethylidene]benzohydrazide compound, the method comprising: adding a solution of methyl-5-bromosalicylic hydrazide in methanol to a solution of 2-acetylpyridine in a mixture of acetonitrile and methanol to obtain a reaction mixture; stirring the reaction mixture; collecting a precipitate by filtration and drying under a vacuum; and obtaining the 5-bromo-2-hydroxy-N′-[(1E)-1-(pyridin-2-yl)ethylidene]benzohydrazide compound.

The present production methods can be further seen by referring to the following Scheme 1:

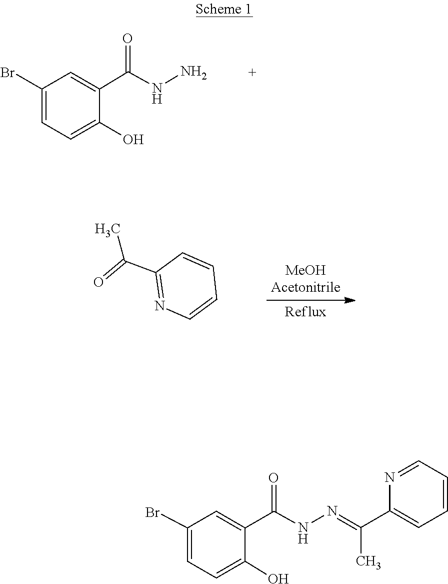

In an embodiment of the present production methods, the reaction mixture may be stirred at a temperature of about 60° C. In another embodiment of the present production methods, the reaction mixture may be stirred for about 18 hours, or at least about 18 hours.

In a further embodiment of the present production methods, the precipitate may be white.

In an embodiment of the present production methods, the methyl-5-bromosalicylic hydrazide and 2-acetylpyridine may be added in an about 1:1 molar ratio.

In an additional embodiment of the present production methods, the 5-bromo-2-hydroxy-N′-[(1E)-1-(pyridin-2-yl)ethylidene]benzohydrazide compound can be obtained in an about 82% yield.

The following examples relate to various methods of manufacturing the specific compounds and application of the same, as described herein. All compound numbers expressed herein are with reference to the synthetic pathway figures shown above.

### EXAMPLES

### Example 1

### Preparation of 5-bromo-2-hydroxy-N′-[(1E)-1-(pyridin-2-yl)ethylidene]benzohydrazide

A solution of methyl-5-bromosalicylic hydrazide (231.05 mg, 1 mmol) in methanol (30 mL) was added to a solution of 2-acetylpyridine (121.14 mg, 1 mmol) in a mixture of acetonitrile and methanol (10 mL) with continuous stirring. The resulting mixtures were stirred at 60° C. over a period of 18 hours (Scheme 1). A white precipitate was formed during the reaction which was collected by filtration and dried under vacuum.

Characterization of the prepared compound (Yield: 82%). IR (KBr disk, cm-1): 3477 (ν OH), 3290 (ν NH), 3088 (ν C—H), 1637 (ν C═O) 1553 (ν C═N). ¹H NMR (400 MHz, DMSO-d6, ppm): δ˜12.3 (br, 1H), δ 11.46 (s, 1H), 8.63 (d, 1H, J=3.6), 8.07 (m, 2H), 7.59 (dd, 1H, J=1.6 Hz), 7.01 (dd, 1H, J=6.8 Hz), 2.44 (s, 3H). ¹³C NMR (100 MHz, DMSO-d6, ppm): 161.03 (C═O), 156.21 (—C—OH), 155.17 (—C═N—), 153.32 (—C═N—), 149.16 (—C═N—), 137.19 (Ar—C), 136.30, 133.20 (Ar—C), 124.73 (Ar—C), 120.91 (Ar—C), 120.67 (Ar—C), 119.85 (Ar—C), 111.29 (Ar—C) 12.31 (—CH₃). DEPT-135: 149.16 (Ar—CH═N—), 137.19 (Ar—CH), 136.30 (Ar—CH), 133.19 (Ar—CH), 124.73 (Ar—CH), 120.91 (Ar—CH), 119.85 (Ar—CH), 12.31 (—CH₃). Mass spectrum (m/z): calcd. 334.16, obs. 334.01 (M+)

### Example 2

### Antimicrobial Study

Initially, compound L was tested against some Gram-negative and Gram-positive bacteria and the fungus strain. The antibacterial activity results demonstrated the compound L to be potentially effective against Salmonella typhi, Staphylococcus aureus, Bacillus Megaterium, and Escherichia coli, with reduced minimal inhibitory concentrations (MICs) of 26, 24, 20 and 17 mm, respectively for the four strains.

Two gram-positive Staphylococcus aureus (cars-2) and Bacillus Megnaterium, two gram-negative Escherichia coli (carsgn-2) and Salmonella Typhi (JCM-1652) bacteria, and two fungal strains Trichoderma harzianum (carsm-2) and Aspergillus niger (carsm-3) were used in this study.

Table 1 shows Diameter of inhibition zones (mm) of the synthesized compounds, Ceftriaxone, and Amphotericin-B against tested bacterial and fungal strains.

| | Gram (+) bacteria | | Gram (−) bacteria | | Fungi | |
|---|---|---|---|---|---|---|
| | S. | B. | E. | S. | T. | A. |
| Compound | aureus | megnaterium | coli | typhi | harzianum | niger |
| L | 24 | 20 | 17 | 26 | 12 | 12 |
| Ceftriaxone | 40.0 | 50.0 | 38.0 | 44.0 | | |
| Amphotericin-B | | | | | 17.0 | 8.0 |
| DMSO | — | — | — | — | — | — |

### Example 3

### Molecular Docking

Molecular docking is a powerful tool in the field of computational and medicinal chemistry. It plays a crucial role in the process of drug discovery and design by giving several quick insights into the binding interactions between a small molecule (ligand) and a target protein (receptor).

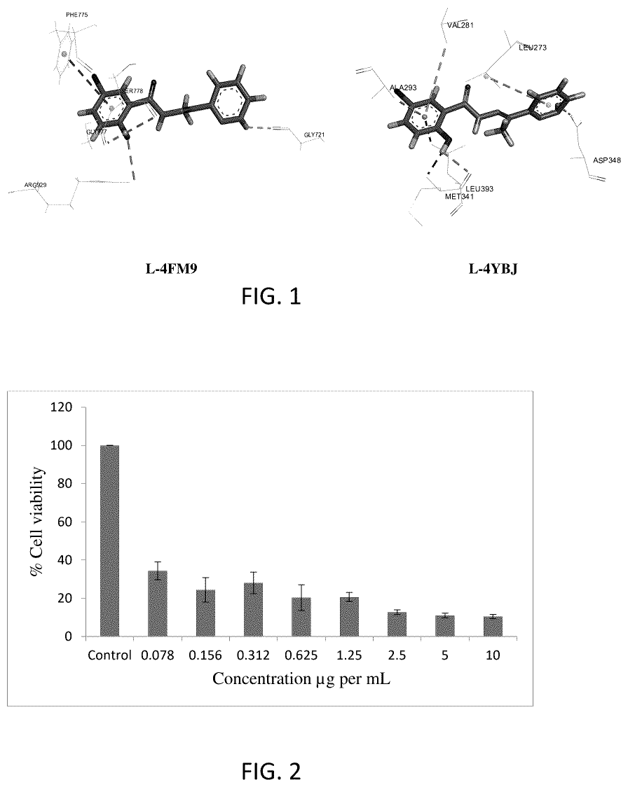

To investigate and compare the anticancer activity of the synthesized compound with experimental data, a docking analysis of L against Liver and lung cancer cell lines was performed. Thus, to get the binding conformations of these compounds, L was docked in the active site of Liver and lung cancer cell lines (PDB ID: 4FM9 and 4YBJ) using the PyRx 0.8. The most stable anchoring conformations of these compounds along with interacting residues are shown in FIG. 1 created with the help of the discovery studio visualizer.

The binding energies for compound L with 4FM9 were found −8.1 kcal·mol⁻¹. In L-4FM9, compound L formed three conventional hydrogen bonds (2.13, 2.69 and 3.02 Å) of O—H—O—C with active site residues of Arg929, Ser778 and Gly777. π-π, π-σ bond interactions were also noticed with Gly721 (2.58 Å) and Phe775 (5.71 Å) as shown in FIG. 1. In L-4YBJ, compound L formed one hydrogen bond (2.45 Å) of O—H—O—C with active site residues of Met341. Results of docking studies revealed that compound L formed bonds to the active site of 4YBJ and showed strong interactions with Ala293, Val281, Met341, Leu393, Asp348, and Leu273. Thus, computational results are in good agreement with in vitro experimental data.

Table 2 shows the calculated binding energies and H-bond count of the targeted Compound L inside the human PDB ID: 4FM9 and 4YBJ active site.

| | Binding | Protein-ligand interaction | | | |
|---|---|---|---|---|---|
| | energy | No of | Amino | | |
| | (kcal/ | H- | acid | Distance | Other interacting |
| System | mol) | bond | residues | (Å) | residues |
| L-4FM9 | −8.1 | 3 | Arg929 | 2.13 | Phe775, Gly721 |
| | | | Ser778 | 2.69 | |
| | | | Gly777 | 3.02 | |
| L-4YBJ | −7.4 | 1 | Met341 | 2.45 | Ala293, Val281, Met341, |
| | | | | | Leu393, Asp348, Leu273 |

### Example 4

### Anti-Cancer Testing

### Description of Cell Line Studies (Lung and Hepatocellular Carcinoma)

MTT (3-(4,5-dimethyl-thiazol-2-yl)-2,5-diphenyltetrazolium bromide) powder, DMEM medium, fetal bovine serum (FBS), Trypsin/EDTA solution, Phosphate buffered saline and Antibiotic-Antimycotic, were purchased from Gibco, Invitrogen (Eugene, OR, USA). Additionally, 96-well plates, T-25 and T-75 cell culture flasks, as well as serological pasture pipettes and micropipette tips were obtained from Corning USA.

### Cell Lines

Two human cancer cell lines were utilized in the cytotoxicity assay. They were lung cancer (A549) and hepatocellular carcinoma (HepG2). A549 and HepG2 cells were cultured in a DMEM medium supplemented with 10% Fetal Bovine Serum (FBS), and 1% Antibiotic-Antimycotic at 37° C., with 5% CO₂ in a 75 cm² tissue culture flask.

### In Vitro Cytotoxicity Studies

The conversion of yellow MTT to purple formazan crystals by mitochondrial dehydrogenase of viable cell enzymes was used to assess the magnitude of cellular cytotoxicity exerted by different concentrations of L as explained earlier Kazi et al. 2023 and modified by Kumar et al. 2022. Briefly, cancer cells were plated in 96-well plates (2×10³ cells/well) in full cell growth medium and incubated for 24 h at 37° C. under a humidified atmosphere of 5% CO₂. The cell medium was then replaced by a cell growth medium containing 5% FBS (DMEM medium), containing different concentrations of L. This experiment was performed in two batches. In the first batch, concentrations of L were 1.95, 3.9, 7.8, 15.6, 31.25, 62.5, 125 and 250 μg/ml with control (without any treatment). In the second batch, concentrations of L were 0.078, 0.156, 0.312, 0.625, 1.25, 2.5, 5.0, and 10.0 μg/mL with control (without any treatment). After 72 hours of incubation, medium in all test and control wells was substituted by 100 L/well of MTT solution (0.5 mg/mL, in PBS) and incubated for a further 3 h at 37° C. Afterwards, the MTT solution was replaced with 100 μL isopropanol/well to dissolve the purple formazan crystals formed at the bottom of the wells, with shaking for at least 2 h at room temperature in dark. Subsequently, the color intensity in the wells was measured at 549 nm with a Bio-Tek microplate reader (ELX 800; Bio-Tek Instruments, Winooski, VT, USA). The results were analyzed in triplicates and the viability percentage was calculated. The data are presented as the percentages of viable cells in the test wells compared to those of the control group. The following equation was used to calculate the cell viability:

$$\% cell viability = [ A 549 nm of treated cells / A 549 nm of control cells ] \times 100$$

Liver and lung cancer cell lines were used to observe the antiproliferative effects of different concentrations of L as a growth-inhibiting study utilizing MTT-based viability assays. The MTT assay is based on mitochondrial activity. Results of the first batch were used as a screening assay and found that the lowest concentration which is 1.96 μg/mL was showing 14.76% cell viability (FIG. 8) for liver cancer cells, which is outstanding cell viability for anti-proliferative activity for new compound. Therefore, we decided to perform MTT further lower concentrations of L. Again, we found that the lowest concentration of L which is 0.078 μg/mL was showing 34.35% cell viability (FIG. 7) for liver cancer cells. The results of both batches are admirable and far better than earlier reported publications. Therefore, we repeated this experiment with lung cancer cell lines and found almost similar results (FIGS. 9 and 10) as were with liver cancer cells.

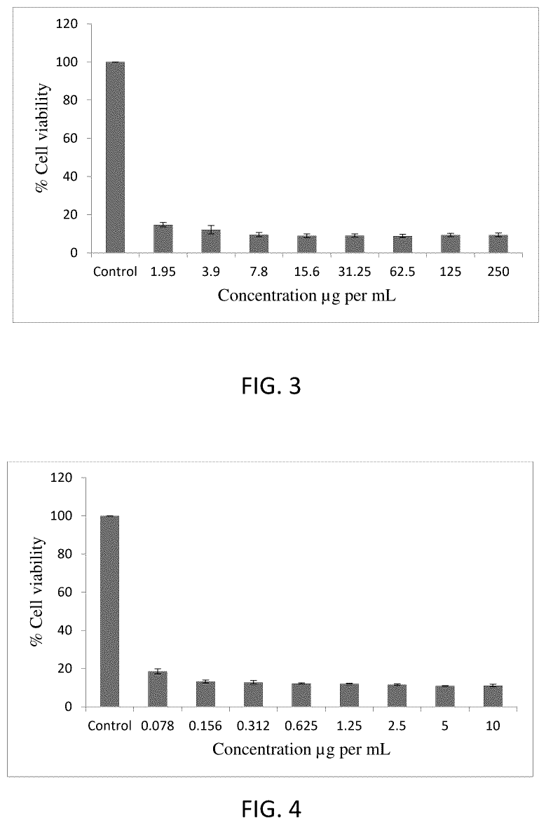

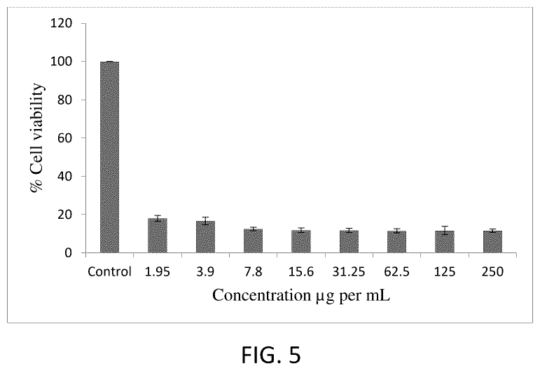

Calculating IC so value was attempted but could not be determined because cell viability % was even less than 50% at 0.078 μg/mL which was the lowest concentration for liver (FIGS. 2 and 3) and lung (FIGS. 4 and 5) cancer less lines.

In FIG. 2, cell viability is shown as % cell viability of 5-bromo-2-hydroxy-N′-[(1E)-1-(pyridin-2-yl)ethylidene]benzohydrazide was 34.35, 24.36, 27.99, 20.27, 20.16, 12.7, 10.95, and 10.39 at doses of 0.078, 0.156, 0.312, 0.625, 1.25, 2.5, 5.0, 10.0 μg/mL, respectively, compared with controls.

In FIG. 3, % cell viability of 5-bromo-2-hydroxy-N′-[(1E)-1-(pyridin-2-yl)ethylidene]benzohydrazide was 14.76, 12.16, 9.57, 8.96, 9.09, 8.86, 9.34, and 9.38 at doses of 1.95, 3.9, 7.8, 15.6, 31.25, 62.5, 125, 250 μg/mL, respectively, compared with controls.

In FIG. 4, % cell viability of 5-bromo-2-hydroxy-N′-[(1E)-1-(pyridin-2-yl)ethylidene]benzohydrazide was 18.61, 13.25, 12.83, 12.3, 12.11, 11.65, 10.96, and 11.12 at doses of 0.078, 0.156, 0.312, 0.625, 1.25, 2.5, 5.0, 10.0 μg/mL, respectively, compared with controls.

In FIG. 5, % cell viability of 5-bromo-2-hydroxy-N′-[(1E)-1-(pyridin-2-yl)ethylidene]benzohydrazide was 17.99, 16.66, 12.47, 11.82, 11.7, 11.45, 11.63, and 11.56 at doses of 1.95, 3.9, 7.8, 15.6, 31.25, 62.5, 125, 250 μg/mL, respectively, compared with controls.

It is to be understood that the 5-bromo-2-hydroxy-N′-[(1E)-1-(pyridin-2-yl)ethylidene]benzohydrazide compound, compositions containing the same, and methods of using and producing the same are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1: A method of making a 5-bromo-2-hydroxy-N′-[(1E)-1-(pyridin-2-yl)ethylidene]benzohydrazide compound having the formula I: 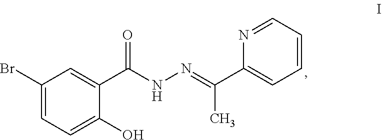 [SMILES: C/C(=N\NC(=O)c1cc(Br)ccc1O)c1ccccn1]
the method comprising:
adding a solution of methyl-5-bromosalicylic hydrazide in methanol to a solution of 2-acetylpyridine in a mixture of acetonitrile and methanol to obtain a reaction mixture;
stirring the reaction mixture;
collecting a precipitate by filtration and drying under a vacuum; and
obtaining the 5-bromo-2-hydroxy-N′-[(1E)-1-(pyridin-2-yl)ethylidene]benzohydrazide compound.

2: The method of making the 5-bromo-2-hydroxy-N′-[(1E)-1-(pyridin-2-yl)ethylidene]benzohydrazide compound of claim 1, wherein the reaction mixture is stirred at a temperature of about 60° C.

3: The method of making the 5-bromo-2-hydroxy-N′-[(1E)-1-(pyridin-2-yl)ethylidene]benzohydrazide compound of claim 1, wherein the reaction mixture is stirred for about 18 hours.

4: The method of making the 5-bromo-2-hydroxy-N′-[(1E)-1-(pyridin-2-yl)ethylidene]benzohydrazide compound of claim 1, wherein the precipitate is white.

5: The method of making the 5-bromo-2-hydroxy-N′-[(1E)-1-(pyridin-2-yl)ethylidene]benzohydrazide compound of claim 1, wherein the methyl-5-bromosalicylic hydrazide and 2-acetylpyridine are added in an about 1:1 molar ratio.

6: The method of making the 5-bromo-2-hydroxy-N′-[(1E)-1-(pyridin-2-yl)ethylidene]benzohydrazide compound of claim 1, wherein the 5-bromo-2-hydroxy-N′-[(1E)-1-(pyridin-2-yl)ethylidene]benzohydrazide compound is obtained in an about 82% yield.
